(19) United States
(12) Patent Application Publication
(10) Pub. No.: US 2026/0125488 A1
(43) Pub. Date: May 7, 2026

(54) Treatment Of IgE-Mediated Diseases With Antibodies That Specifically Bind CD38

(71) Applicant: UMC Utrecht Holding B.V., Utrecht (NL)

(72) Inventor: Henny G. Otten, Utrecht (NL)

(21) Appl. No.: 19/437,288

(22) Filed: Dec. 30, 2025

Related U.S. Application Data
(63) Continuation of application No. 18/329,443, filed on Jun. 5, 2023, now Pat. No. 12,528,878.
Application No. 18/329,443 is a continuation of application No. 16/312,133, now abandoned.
(60) Provisional application No. 62/355,526, filed on Jun. 28, 2016.

(51) Int. Cl. C07K 16/28; A61K 31/454; A61K 31/573; A61K 38/47; A61K 39/00; A61K 39/395; A61P 37/08

(52) U.S. Cl. CPC C07K 16/2896; A61K 31/454; A61K 31/573; A61K 38/47; A61K 39/395; A61K 39/39533; A61K 39/39541; A61P 37/08; C12Y 302/01035; A61K 2039/505; C07K 2317/24; C07K 2317/56; C07K 2317/732

(57) ABSTRACT

The present invention relates to treatment of IgE-mediated disease with antibodies that specifically bind CD38.

### RELATED APPLICATIONS

This application is a continuation of U.S. application Ser. No. 18/329,443, filed on Jun. 5, 2023, which is a continuation of U.S. application Ser. No. 16/312,133, filed on Dec. 20, 2018, now abandoned, which is the U.S. National Stage of International Application No. PCT/EP2017/066063, filed on Jun. 28, 2017, published in English, which claims the benefit of U.S. Provisional Application No. 62/355,526, filed on Jun. 28, 2016. The entire teachings of the above applications are incorporated herein by reference.

### INCORPORATION BY REFERENCE OF MATERIAL IN XML

This application incorporates by reference the Sequence Listing contained in the following extensible Markup Language (XML) file being submitted concurrently herewith:

File name: 01482021007.xml; created Dec. 17, 2025, 29,598 Bytes in size.

### FIELD OF THE INVENTION

The present invention relates to treatment of IgE-mediated disease with antibodies that specifically bind CD38.

### BACKGROUND OF THE INVENTION

A large and increasing proportion of the population in industrialized countries suffer from allergies. The current estimate for this debilitating condition is one in three people and a large proportion of this population is notably children. The pathogenesis of allergy is mediated by dysregulated triggering of IgE-mediated immune responses following repeated encounters with environmental antigens. IgE-mediated allergies are triggered by binding of IgE to the high affinity IgE receptor (FcERI), which is expressed on effector mast cells, basophils and activated eosinophils. As a result of these high affinity interactions, stable FcERI: IgE complexes are displayed on the surface of effector cells. Exposure to allergens leads to cross-linking and eventually clustering of IgE: FcERI complexes, thus triggering effector cell activation, degranulation and release of stored pro-allergenic mediators that leads to the initiation of an allergic response.

Common environmental allergens which induce anaphylactic hypersensitivity are found in pollen, foods, house dust mites, animal danders, fungal spores and insect venoms. Atopic allergy is associated with anaphylactic hypersensitivity and includes the disorders, e.g., asthma, allergic rhinitis and conjunctivitis (hay fever), eczema, urticaria and food allergies. Further, an allergic reaction may lead to a dangerous life-threatening condition such as anaphylactic shock, which may be provoked by insect bites.

For example, food allergy affects millions of people and is responsible for substantial morbidity, impaired quality of life and costs to the individual, family and society (Mills et al., Allergy 2007; 62:717-22. doi: 10.1111/j.1398-9995.2007.01425.x.). Recent studies estimate that the prevalence of food allergy in the general population is around 5% for adults and 8% for children (Sicherer et al., J Allergy Clin Immunol 2014; 133:291-307.e5. doi: 10.1016/j.jaci.2013.11.020.). The economic burden of food allergy in allergic children in the US are estimated $4184 per year per child (Gupta R et al., JAMA Pediatr 2013; 167:1026. doi: 10.1001/jamapediatrics.2013.2376). Allergic reactions can vary from mild symptoms limited to the oral cavity and skin, to severe respiratory and cardiovascular symptoms that can be potentially fatal. Emergency management of food allergic reactions includes administration of epinephrine, corticosteroids and antihistamines. With no curative treatment available, strict avoidance of the eliciting allergens is often necessary. Still, accidental ingestion occurs, often leading to hospital admission and treatment in an intensive care unit.

In addition to allergies, IgE plays a role in autoimmune disorders contributing to their pathogenesis (Ettinger et al., Autoimmunity 50:25-35, 2017; Holgate, World Allergy Organization Journal 7:17, 2014).

A need exists for treatment options for IgE-mediated diseases such as allergies and autoimmune diseases.

### SUMMARY OF THE INVENTION

The invention provides for a method of treating an IgE-mediated disease, comprising administering to a subject in need thereof an antibody that specifically binds CD38 for a time sufficient to treat the IgE-mediated disease.

### BRIEF DESCRIPTION OF THE DRAWINGS

FIG. 1 shows that total IgE and timothy grass and house dust mite-specific IgE is reduced in a multiple myeloma patient treated with DARZALEX™ (daratumumab) over time.

### DETAILED DESCRIPTION OF THE INVENTION

“CD38” refers to the human CD38 protein (synonyms: ADP-ribosyl cyclase 1, cADPr hydrolase 1, cyclic ADP-ribose hydrolase 1). Human CD38 has an amino acid sequence shown in GenBank accession number NP_001766 and in SEQ ID NO: 1. It is well known that CD38 is a single pass type II membrane protein with amino acid residues 1-21 representing the cytosolic domain, amino acid residues 22-42 representing the transmembrane domain, and residues 43-300 representing the extracellular domain of CD38.

SEQ ID NO: 1
MANCEFSPVSGDKPCCRLSRRAQLCLGVSILVLILVVVLA
VVVPRWRQQWSGPGTTKRFPETVLARCVKYTEIHPEMRHV
DCQSVWDAFKGAFISKHPCNITEEDYQPLMKLGTQTVPCN
KILLWSRIKDLAHQFTQVQRDMFTLEDTLLGYLADDLTWC
GEFNTSKINYQSCPDWRKDCSNNPVSVFWKTVSRRFAEAA
CDVVHVMLNGSRSKIFDKNSTFGSVEVHNLQPEKVQTLEA
WVIHGGREDSRDLCQDPTIKELESIISKRNIQFSCKNIYR
PDKFLQCVKNPEDSSCTSEI

“Antibodies” as used herein is meant in a broad sense and includes immunoglobulin molecules including monoclonal antibodies including murine, human, humanized and chimeric monoclonal antibodies, antibody fragments, bispecific or multispecific antibodies, dimeric, tetrameric or multimeric antibodies, single chain antibodies, domain antibodies and any other modified configuration of the immunoglobulin molecule that comprises an antigen binding site of the required specificity.

Immunoglobulins may be assigned to five major classes, namely IgA, IgD, IgE, IgG and IgM, depending on the heavy chain constant domain amino acid sequence. IgA and IgG are further sub-classified as the isotypes IgAQ1, IgA2, IgG1, IgG2, IgG3 and IgG4. Antibody light chains of any vertebrate species can be assigned to one of two clearly distinct types, namely kappa (κ) and lambda (λ), based on the amino acid sequences of their constant domains.

“Antibody fragments” refers to a portion of an immunoglobulin molecule that retains the heavy chain and/or the light chain antigen binding site, such as heavy chain complementarity determining regions (HCDR) 1, 2 and 3, light chain complementarity determining regions (LCDR) 1, 2 and 3, a heavy chain variable region (VH), or a light chain variable region (VL). Antibody fragments include a Fab fragment, a monovalent fragment consisting of the VL, VH, CL and CH1 domains; a F (ab) 2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a domain antibody (dAb) fragment (Ward et al., Nature 341:544-6, 1989), which consists of a VH domain or a VL domain. VH and VL domains may be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv (scFv) or diabody; described for example in Intl. Pat. Publ. Nos. WO1998/44001, WO1988/01649, WO1994/13804, and WO1992/01047. These antibody fragments are obtained using well known techniques known to those of skill in the art, and the fragments are screened for utility in the same manner as are full length antibodies.

“Isolated antibody” refers to an antibody or antibody fragment that is substantially free of other antibodies having different antigenic specificities (e.g., an isolated antibody specifically binding CD38 is substantially free of antibodies that specifically bind antigens other than human CD38). An isolated antibody that specifically binds CD38, however, may have cross-reactivity to other antigens, such as orthologs of human CD38, such as Macaca fascicularis (cynomolgus) CD38. In case of a bispecific antibody, the bispecific antibody specifically binds two antigens of interest, and is substantially free of antibodies that specifically bind antigens other that the two antigens of interest. Moreover, an isolated antibody may be substantially free of other cellular material and/or chemicals. “Isolated antibody” encompasses antibodies that are isolated to a higher purity, such as antibodies that are 80%, 81%, 82%, 83%, 84%, 85%, 86%, 87%, 88%, 89%, 90%, 91%, 92%, 93%, 94%, 95%, 96%, 97%, 98%, 99% or 100% pure.

An antibody variable region consists of a “framework” region interrupted by three “antigen binding sites”. The antigen binding sites are defined using various terms: Complementarity Determining Regions (CDRs), three in the VH (HCDR1, HCDR2, HCDR3) and three in the VL (LCDR1, LCDR2, LCDR3) are based on sequence variability (Wu and Kabat J Exp Med 132:211-50, 1970; Kabat et al Sequences of Proteins of Immunological Interest, 5th Ed. Public Health Service, National Institutes of Health, Bethesda, Md., 1991); “Hypervariable regions”, “HVR”, or “HV”, three in the VH (H1, H2, H3) and three in the VL (L1, L2, L3) refer to the regions of antibody variable domains which are hypervariable in structure as defined by Chothia and Lesk (Chothia and Lesk Mol Biol 196:901-17, 1987). Other terms include “IMGT-CDRs” (Lefranc et al., Dev Comparat Immunol 27:55-77, 2003) and “Specificity Determining Residue Usage” (SDRU) (Almagro, Mol Recognit 17:132-43, 2004). The International ImMunoGeneTics (IMGT) database (http://www_imgt_org) provides a standardized numbering and definition of antigen-binding sites. The correspondence between CDRs, HVs and IMGT delineations is described in Lefranc et al., Dev Comparat Immunol 27:55-77, 2003.

“Chothia residues” as used herein are the antibody VL and VH residues numbered according to Al-Lazikani (Al-Lazikani et al., J Mol Biol 273:927-48, 1997).

“Framework” or “framework sequences” are the remaining sequences of a variable region other than those defined to be antigen binding sites. Because the antigen binding sites can be defined by various terms as described above, the exact amino acid sequence of a framework depends on how the antigen-binding site was defined.

“Humanized antibody” refers to an antibody in which the antigen binding sites are derived from non-human species and the variable region frameworks are derived from human immunoglobulin sequences. Humanized antibodies may include substitutions in the framework regions so that the framework may not be an exact copy of expressed human immunoglobulin or germline gene sequences.

“Human antibody” refers to an antibody having heavy and light chain variable regions in which both the framework and the antigen binding sites are derived from sequences of human origin and is optimized to have minimal immune response when administered to a human subject. If the antibody contains a constant region, the constant region also is derived from sequences of human origin.

A human antibody comprises heavy or light chain variable regions that are “derived from” sequences of human origin wherein the variable regions of the antibody are obtained from a system that uses human germline immunoglobulin or rearranged immunoglobulin genes. Such systems include human immunoglobulin gene libraries displayed on phage, and transgenic non-human animals such as mice carrying human immunoglobulin loci as described herein. A “human antibody” may contain amino acid differences when compared to the human germline immunoglobulin or rearranged immunoglobulin genes due to differences between the systems used to obtain the antibody and human immunoglobulin loci, introduction of somatic mutations or intentional introduction of substitutions in the framework or antigen binding site, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98%, 99% or 100% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., J Mol Biol 296:57-86, 2000, or synthetic HCDR3 incorporated into human immunoglobulin gene libraries displayed on phage, for example as described in Shi et al., J Mol Biol 397:385-96, 2010 and Intl. Pat. Publ. No. WO2009/085462.

Human antibodies derived from human immunoglobulin sequences may be generated using systems such as phage display incorporating synthetic CDRs and/or synthetic frameworks, or can be subjected to in vitro mutagenesis to improve antibody properties, resulting in antibodies that do not naturally exist within the human antibody germline repertoire in vivo.

Antibodies in which antigen binding sites are derived from a non-human species are not included in the definition of human antibody.

“Recombinant antibody” includes all antibodies that are prepared, expressed, created or isolated by recombinant means, such as antibodies isolated from an animal (e.g., a mouse or a rat) that is transgenic or transchromosomal for human immunoglobulin genes or a hybridoma prepared therefrom (described further below), antibodies isolated from a host cell transformed to express the antibody, antibodies isolated from a recombinant, combinatorial antibody library, and antibodies prepared, expressed, created or isolated by any other means that involve splicing of human immunoglobulin gene sequences to other DNA sequences, or antibodies that are generated in vitro using Fab arm exchange such as bispecific antibodies.

“Monoclonal antibody” refers to a preparation of antibody molecules of single molecular composition. A monoclonal antibody composition displays a single binding specificity and affinity for a particular epitope, or in a case of a bispecific monoclonal antibody, a dual binding specificity to two distinct epitopes. “Monoclonal antibody” therefore refers to an antibody population with single amino acid composition in each heavy and each light chain, except for possible well known alterations such as removal of C-terminal lysine from the antibody heavy chain or alterations due to post-translational modification(s) of amino acids, such as methionine oxidation or asparagine or glutamine deamidation. Monoclonal antibodies may have heterogeneous glycosylation within the antibody population. Monoclonal antibody may be monospecific or multispecific, or monovalent, bivalent or multivalent. A bispecific antibody is included in the term monoclonal antibody.

“Epitope” means a portion of an antigen to which an antibody specifically binds. Epitopes usually consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or noncontiguous amino acids that form a conformational spatial unit. For a noncontiguous epitope, amino acids from differing portions of the linear sequence of the antigen come in close proximity in 3-dimensional space through the folding of the protein molecule.

“Variant” refers to a polypeptide or a polynucleotide that differs from a reference polypeptide or a reference polynucleotide by one or more modifications for example, substitutions, insertions or deletions.

“In combination with” means that two or more therapeutics are administered to a subject together in a mixture, concurrently as single agents or sequentially as single agents in any order. In general, each agent will be administered at a dose and/or on a time schedule determined for that agent.

“Treat” or “treatment” refers to therapeutic treatment wherein the object is to slow down (lessen) an undesired physiological change or disease, or to provide a beneficial or desired clinical outcome during treatment. Beneficial or desired clinical outcomes include alleviation of symptoms, diminishment of extent of disease, stabilized (i.e., not worsening) state of disease, delay or slowing of disease progression, amelioration or palliation of the disease state. Those in need of treatment include those subjects already with the undesired physiological change or disease as well as those subjects prone to have the physiological change or disease.

“Therapeutically effective amount” refers to an amount effective, at dosages and for periods of time necessary, to achieve a desired therapeutic result. A therapeutically effective amount may vary according to factors such as the disease severity, age, sex, and weight of the individual, and the ability of a therapeutic or a combination of therapeutics to elicit a desired response in the individual. Exemplary indicators of an effective therapeutic or combination of therapeutics include, for example, improved well-being of the subject, reduction in symptoms of the disease, such as reduction in sneezing, coughing, sinus congestion, mucus production in the sinuses (rhinitis) or lungs (asthma), itching, swelling, and/or decreased IgE levels in a subject.

“Patient” includes any human or nonhuman animal. “Nonhuman animal” includes all vertebrates, e.g., mammals and non-mammals, such as nonhuman primates, sheep, dogs, cats, horses, cows chickens, amphibians, reptiles, etc. The terms “patient” and “subject” are used interchangeably.

“Specific binding” or “specifically binds” or “binds” refers to an antibody binding to an antigen or an epitope within the antigen with greater affinity than for other antigens. Typically, the antibody binds to the antigen or the epitope within the antigen with an equilibrium dissociation constant (K_{D})) of about 1×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred-fold less than its K_{D} for binding to a non-specific antigen (e.g., BSA, casein). The dissociation constant may be measured using standard procedures. Antibodies that specifically bind to the antigen or the epitope within the antigen may, however, have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset). While a monospecific antibody specifically binds one antigen or one epitope, a bispecific antibody specifically binds two distinct antigens or two distinct epitopes.

“An IgE-mediated disease” refers to a disease that is mediated, at least in part, by an increase in the level of IgE in a subject. “Increase in the level of IgE” refers to the level of total IgE of >2 kU/L and/or the level of allergen specific IgE of ≥0.35 kU/L using ImmunoCAP assay (ThermoFisher, Uppsala, Sweden) using methodology described herein and according to the manufacturer's instructions. IgE-mediated diseases include disorders associated with increased IgE levels or activity in which atypical symptoms may manifest due to levels of IgE locally and/or systemically in the body even if the threshold of IgE of >2 kU/L is not achieved systemically.

The current invention is based, at least in part, on the identification that treatment of a subject with an antibody that specifically binds CD38 reduces total IgE and allergen-specific IgE in the subject over time. By not wishing to be bound by any particular theory, it is believed that the antibody that specifically binds CD38 mediates killing of the B cells expressing and/or secreting IgE.

Allergic diseases are conventionally described as IgE-mediated diseases. Clinical manifestations of allergic diseases include allergic asthma, allergic rhinitis, atopic dermatitis, food hypersensitivity and anaphylactic reactions to insect bites or drugs. Allergic diseases are caused by hypersensitivity reactions of the immune system to specific allergens (such as pollen, stings, drugs, or food). The most severe form of an allergic disorder is anaphylactic shock.

The specific immune response that defines allergy is characterized by the presence of immunoglobulin type E (IgE) directed to an allergen (sensitization). After sensitization, exposure to the allergen induces crosslinking of IgE bound to mast cells and basophils causing a wide release of vasoactive mediators, such as histamine. Therefore, therapy directed to IgE has been a research focus for several years. Therapy using monoclonal antibodies against IgE (XolairR (omalizumab)) has proven to be effective in allergic asthma [4], with effectiveness also being researched in other (atopic) diseases, such as allergic rhinitis, and atopic dermatitis (Baena-Cagnani C E et al., Curr Opin Allergy Clin Immunol 2014; 14:149-54. doi: 10.1097/ACI.0000000000000044). Furthermore, the role of XolairR (omalizumab) in (oral) immunotherapy (IT) for food allergy is actively investigated, with first results demonstrating a decrease in adverse effects during treatment, similar to previous studies on IT for aeroallergens (Wood R A et al., J Allergy Clin Immunol 2015. doi: 10.1016/j.jaci.2015.10.005). Another monoclonal antibody currently under investigation is quilizumab, which targets membrane IgE-expressing cells and thereby disrupting IgE production (Gauvreau G M et al., Sci Transl Med 2014; 6: 243ra85. doi: 10.1126/scitranslmed.3008961).

A different method of targeting IgE may be by depleting plasma cells that produce (specific) IgE. The effects of B-cell depletion on IgE levels and clinical symptoms of atopic diseases have been studied before. RituxanR (rituximab), for example, did not significantly decrease serum IgE levels compared to placebo at three or six months in patients with Idiopathic Thrombocytopenia Purpura (Dasgupta A et al., Allergy Asthma Clin Immunol 2013; 9:39. doi: 10.1186/1710-1492-9-39). VELCADE® (bortezomib), targeting (malignant) plasma cells via proteasome inhibition, is also being investigated as a treatment option in (auto) antibody mediated diseases (Rosenberg A S et al., Clin Immunol 2016. doi: 10.1016/j.clim.2016.02.009). In a mouse model for chronic asthma, bortezomib treatment reduced specific IgE levels (Wegmann M et al., Int Arch Allergy Immunol 2012; 158:43-53. doi: 10.1159/000330103).

The invention provides a method of treating an IgE-mediated disease, comprising administering to a subject in need thereof a therapeutically effective amount of an antibody that specifically binds CD38 for a time sufficient to treat the IgE-mediated disease.

The invention also provides for an antibody that specifically binds CD38 for use in treating a subject having an IgE-mediated disease.

The invention also provides use of an antibody that specifically binds CD38 in the manufacture of a medicament for the treatment of an IgE-mediated disease.

The invention also provides use of an antibody that specifically binds CD38 in the preparation of a pharmaceutical composition for the treatment of an IgE-mediated disease.

In some embodiments, the IgE-mediated disease is an allergic response to an allergen.

Exemplary allergens include airborne allergens, such as those of house dust mite, pets and pollens, for example house dust mites allergens obtained from Dermatophagoides spp or D. pteronyssinus, D. farinae and D. microceras, Euroglyphus maynei or Blomia sp., allergens from insects present in cockroach or Hymenoptera, allergens from pollen, such as pollens of tree, grass and weed, allergens from animals, especially in cat, dog, horse and rodent, allergens from fungi, such as from Aspergillus, Altemaria or Cladosporium, and occupational allergens including animal and plant antigens as well as drugs, detergents, metals and immunoenhancers such as isocyanates and allergens present in products such as latex or amylase.

Exemplary allergens also include ingested allergens responsible for food hypersensitivity, such as fruits, vegetables and milk, such as food allergens present in peanuts, fish e.g. codfish, egg white, crustacean e.g. shrimp, milk e.g. cow's milk, wheat, cereals, fruits of the Rosacea family (apple, plum, strawberry), vegetables of the Liliacea, Cruciferae, Solanaceae and Umbelliferae families, tree nuts, sesame, peanut, soybean and other legume family allergens, spices, melon, avocado, mango, fig, banana.

Non-antigen specific stimuli that can result in an IgE-mediated reaction include infection, irritants such as smoke, combustion fumes, diesel exhaust particles and sulphur dioxide, exercise, cold and emotional stress.

Specific hypersensitivity reactions in atopic and nonatopic individuals with a certain genetic background may result from exposure to proteins in foods (e.g., legumes, peanuts), venom (e.g., insect, snake), vaccines, hormones, antiserum, enzymes, latex, antibiotics, muscle relaxants, vitamins, cytotoxins, opiates, and polysaccharides such as dextrin, iron dextran and polygeline.

In some embodiments, the allergen is pollen, a dust mite, a food allergen, a plant allergen, animal dander, insect stings, a fungus, a spore, a mold, latex, or a drug.

IgE has also been associated with pathogenic mechanisms of inflammation and autoimmunity. In addition to SLE and RA, IgE autoantibodies have been detected in dermatological autoimmune disorders such as bullous pemphigoid (BP) and chronic spontaneous urticaria (CSU), in systemic sclerosis, thyroiditis, multiple sclerosis and atopic dermatitis. Immune complexes of IgE autoantibodies and autoantigens may trigger mast cell and basophil degranulation and induce IFNα, TNF and IL-6 production by dendritic cells (DC). IgE can promote antigen cross-presentation triggering both CD4 and CD8 T cell responses, and drive B cell expansion and plasma cell differentiation via DC activation. (reviewed in Ettinger et al., Autoimmunity 50:25-35, 2017; Holgate, World Allergy Organization Journal 7:17, 2014). Xolair® (Omalizumab), an anti-IgE antibody has been reported to exhibit efficacy or is being investigated in at least non-allergic asthma, Churg-Strauss Syndrome, allergic rhinitis, atopic dermatitis, nasal polyposis, food allergy, chronic urticaria and angioedema, Kimura's disease, mastocytosis, anaphylaxis, systemic lupus erythematosus, Sjogren's Syndrome (Holgate, World Allergy Organization Journal 7:17, 2014; ClinicalTrials registry). Therefore, it can be expected that an anti-CD38 antibody depleting IgE producing cells would be efficacious in these diseases.

IgE-mediated diseases include, asthma, atopic dermatitis, allergic rhinitis, fibrosis (e.g., pulmonary fibrosis, such as IPF), allergic asthma, food allergy, anaphylaxis, contact dermatitis, allergic gastroenteropathy, allergic bronchopulmonary aspergillosis, allergic purpura (Henoch-Schonlein), ataxia-telangiectasia, Churg-Strauss Syndrome, eczema, enteritis, gastroenteropathy, graft-versus-host reaction, hyper-IgE (Job's) syndrome, hypersensitivity (e.g., anaphylactic hypersensitivity, candidiasis, vasculitis), IgE myeloma, inflammatory bowel disease (e.g., Crohn's disease, ulcerative colitis, indeterminate colitis and infectious colitis), mucositis (e.g., oral mucositis, gastrointestinal mucositis, nasal mucositis and proctitis), necrotizing enterocolitis and esophagitis, parasitic diseases (e.g., trypanosomiasis), hypersensitivity vasculitis, urticaria, cholinergic urticaria, Wiskott-Aldrich syndrome, lupus, type I diabetes, Kimura's disease, nasal polyposis, eosinophilic gastroenteritis, eosinophilic otitis media, latex allergy, Sjogren's syndrome and rheumatoid arthritis, mastocytosis, cutaneous mastocytosis, chronic or recurrent idiopathic angioedema.

In some embodiments, lupus may be systemic lupus erythematosus (SLE), discoid lupus erythematosus, subacute cutaneous lupus erythematosus, neonatal lupus or drug-induced lupus.

Additionally, disorders that may be treatable by lowering IgE levels, regardless of whether the disorders themselves are associated with elevated IgE are within the scope of IgE-mediated disease.

In some embodiments, the IgE-mediated disease is allergic asthma.

In some embodiments, the IgE-mediated disease is urticaria.

In some embodiments, the IgE-mediated disease is angioedema.

In some embodiments, the IgE-mediated disease is food allergy.

In some embodiments, the IgE-mediated disease is atopic dermatitis.

In some embodiments, the IgE-mediated disease is anaphylaxis.

In some embodiments, the IgE-mediated disease is cutaneous mastocytosis.

In some embodiments, the IgE-mediated disease is allergic rhinitis.

In some embodiments, the IgE-mediated disease is nasal polyposis.

In some embodiments, the IgE-mediated disease is Kimura's disease.

In some embodiments, the IgE-mediated disease is eosinophilic otitis media.

In some embodiments, the IgE-mediated disease is eosinophilic gastroenteritis.

In some embodiments, the IgE-mediated disease is latex allergy.

In some embodiments, the IgE-mediated disease is bronchopulmonary allergic aspergillosis.

In some embodiments, the IgE-mediated disease is bullous pemphigoid (BP).

In some embodiments, the IgE-mediated disease is systemic lupus erythematosus (SLE).

In some embodiments, the IgE-mediated disease is rheumatoid arthritis (RA).

In some embodiments, the IgE-mediated disease is acute or chronic.

In some embodiments, the IgE-mediated disease is an acute disease.

In some embodiments, the IgE-mediated disease is a chronic disease.

In some embodiments, the antibody that specifically binds CD38 competes for binding to CD38 with an antibody comprising a heavy chain variable region (VH) of SEQ ID NO: 4 and a light chain variable region (VL) of SEQ ID NO: 5.

In some embodiments, the antibody that specifically binds CD38 binds at least to the region SKRNIQFSCKNIYR (SEQ ID NO: 2) and the region EKVQTLEAWVIHGG (SEQ ID NO: 3) of CD38 (SEQ ID NO: 1).

In some embodiments, the antibody that specifically binds CD38 comprises a heavy chain complementarity determining region 1 (HCDR1), a HCDR2 and a HCDR3 amino acid sequences of SEQ ID NOs: 6, 7 and 8, respectively.

In some embodiments, the antibody that specifically binds CD38 comprises a light chain complementarity determining region 1 (LCDR1), a LCDR2 and a LCDR3 amino acid sequences of SEQ ID NOs: 9, 10 and 11, respectively.

In some embodiments, the antibody that specifically binds CD38 comprises the HCDR1, the HCDR2, the HCDR3, the LCDR1, the LCDR2, and the LCDR3 amino acid sequences of SEQ ID NOs: 6, 7, 8, 9, 10 and 11, respectively.

In some embodiments, the antibody that specifically binds CD38 comprises the VH that is 95%, 96%, 97%, 98%, 99% or 100% identical to the amino acid sequence of SEQ ID NO: 4 and the VL that is 95%, 96%, 97%, 98%, 99% or 100% identical to the amino acid sequence of SEQ ID NO: 5.

In some embodiments, the antibody that specifically binds CD38 comprises the VH of SEQ ID NO: 4 and the VL of SEQ ID NO: 5.

In some embodiments, the antibody that specifically binds CD38 comprises a heavy chain that is 95%, 96%, 97%, 98%, 99% or 100% identical to the amino acid sequence of SEQ ID NO: 12 and a light chain that is 95%, 96%, 97%, 98%, 99% or 100% identical to the amino acid sequence of SEQ ID NO: 13.

In some embodiments, the antibody that specifically binds CD38 comprises the heavy chain of SEQ ID NO: 12 and the light chain of SEQ ID NO: 13.

An exemplary antibody that specifically binds CD38 that may be used in the methods of the invention is DARZALEX™ (daratumumab). DARZALEX™ (daratumumab) comprises the heavy chain variable region (VH) and the light chain variable region (VL) amino acid sequences shown in SEQ ID NO: 4 and 5, respectively, heavy chain CDRs HCDR1, HCDR2 and HCDR3 of SEQ ID NOs: 6, 7 and 8, respectively, and light chain CDRs LCDR1, LCDR2 and LCDR3 of SEQ ID NOs: 9, 10 and 11, respectively, and is of IgG1/K subtype and described in U.S. Pat. No. 7,829,673. DARZALEX™ (daratumumab) heavy chain amino acid sequence is shown in SEQ ID NO: 12 and light chain amino acid sequence shown in SEQ ID NO: 13.

SEQ ID NO: 2
SKRNIQFSCKNIYR
SEQ ID NO: 3
EKVQTLEAWVIHGG
SEQ ID NO: 4
EVQLLESGGGLVQPGGSLRLSCAVSGFTFNSFAMSWVRQA
PGKGLEWVSAISGSGGGTYYADSVKGRFTISRDNSKNTLY
LQMNSLRAEDTAVYFCAKDKILWFGEPVFDYWGQGTLVTV
SS
SEQ ID NO: 5
EIVLTQSPATLSLSPGERATLSCRASQSVSSYLAWYQQKP
GQAPRLLIYDASNRATGIPARFSGSGSGTDFTLTISSLEP
EDFAVYYCQQRSNWPPTFGQGTKVEIK
SEQ ID NO: 6
SFAMS
SEQ ID NO: 7
AISGSGGGTYYADSVKG
SEQ ID NO: 8
DKILWFGEPVFDY
SEQ ID NO: 9
RASQSVSSYLA
SEQ ID NO: 10
DASNRAT
SEQ ID NO: 11
QQRSNWPPTF
SEQ ID NO: 12
EVQLLESGGGLVQPGGSLRLSCAVSGFTFNSFAMSWVRQA
PGKGLEWVSAISGSGGGTYYADSVKGRFTISRDNSKNTLY
LQMNSLRAEDTAVYFCAKDKILWFGEPVFDYWGQGTLVTV
SSASTKGPSVFPLAPSSKSTSGGTAALGCLVKDYFPEPVT
VSWNSGALTSGVHTFPAVLQSSGLYSLSSVVTVPSSSLGT
QTYICNVNHKPSNTKVDKRVEPKSCDKTHTCPPCPAPELL
GGPSVFLFPPKPKDTLMISRTPEVTCVVVDVSHEDPEVKF
NWYVDGVEVHNAKTKPREEQYNSTYRVVSVLTVLHQDWLN
GKEYKCKVSNKALPAPIEKTISKAKGQPREPQVYTLPPSR
EEMTKNQVSLTCLVKGFYPSDIAVEWESNGQPENNYKTTP
PVLDSDGSFFLYSKLTVDKSRWQQGNVFSCSVMHEALHNH
YTQKSLSLSPGK
SEQ ID NO: 13
EIVLTQSPATLSLSPGERATLSCRASQSVSSYLAWYQQKP
GQAPRLLIYDASNRATGIPARFSGSGSGTDFTLTISSLEP
EDFAVYYCQQRSNWPPTFGQGTKVEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSFNRGEC

Other exemplary antibodies that specifically bind CD38 that may be used in the methods of the invention are:

mAb003 comprising the VH and the VL sequences of SEQ ID NOs: 14 and 15, respectively and described in U.S. Pat. No. 7,829,673. The VH and the VL of mAb003 may be expressed as IgG1/κ.

SEQ ID NO: 14
QVQLVQSGAEVKKPGSSVKVSCKASGGTFSSYAFSWVRQA
PGQGLEWMGRVIPFLGIANSAQKFQGRVTITADKSTSTAY
MDLSSLRSEDTAVYYCARDDIAALGPFDYWGQGTLVTVSS
AS
SEQ ID NO: 15
DIQMTQSPSSLSASVGDRVTITCRASQGISSWLAWYQQKP
EKAPKSLIYAASSLQSGVPSRFSGSGSGTDFTLTISSLQP
EDFATYYCQQYNSYPRTFGQGTKVEIK

mAb024 comprising the VH and the VL sequences of SEQ ID NOs: 16 and 17, respectively, described in U.S. Pat. No. 7,829,673. The VH and the VL of mAb024 may be expressed as IgG1/κ.

SEQ ID NO: 16
EVQLVQSGAEVKKPGESLKISCKGSGYSFSNYWIGWVRQM
PGKGLEWMGIIYPHDSDARYSPSFQGQVTFSADKSISTAY
LQWSSLKASDTAMYYCARHVGWGSRYWYFDLWGRGTLVTV
SS
SEQ ID NO: 17
EIVLTQSPATLSLSPGERATLSCRASQSVSSYLAWYQQKP
GQAPGLLIYDASNRASGIPARFSGSGSGTDFTLTISSLEP
EDFAVYYCQQRSNWPLTFGGGTKVEIK

MOR-202 (MOR-03087) comprising the VH and the VL sequences of SEQ ID NOs: 18 and 19, respectively, described in U.S. Pat. No. 8,088,896. The VH and the VL of MOR-202 may be expressed as IgG1/κ.

SEQ ID NO: 18
QVQLVESGGGLVQPGGSLRLSCAASGFTFSSYYMNWVRQA
PGKGLEWVSGISGDPSNTYYADSVKGRFTISRDNSKNTLY
LQMNSLRAEDTAVYYCARDLPLVYTGFAYWGQGTLVTVSS
SEQ ID NO: 19
DIELTQPPSVSVAPGQTARISCSGDNLRHYYVYWYQQKPG
QAPVLVIYGDSKRPSGIPERFSGSNSGNTATLTISGTQAE
DEADYYCQTYTGGASLVFGGGTKLTVLGQ

Isatuximab; comprising the VH and the VL sequences of SEQ ID NOs: 20 and 21, respectively, described in U.S. Pat. No. 8,153,765. The VH and the VL of Isatuximab may be expressed as IgG1/κ.

SEQ ID NO: 20
QVQLVQSGAEVAKPGTSVKLSCKASGYTFTDYWMQWVKQR
PGQGLEWIGTIYPGDGDTGYAQKFQGKATLTADKSSKTVY
MHLSSLASEDSAVYYCARGDYYGSNSLDYWGQGTSVTVSS
SEQ ID NO: 21:
DIVMTQSHLSMSTSLGDPVSITCKASQDVSTVVAWYQQKP
GQSPRRLIYSASYRYIGVPDRFTGSGAGTDFTFTISSVQA
EDLAVYYCQQHYSPPYTFGGGTKLEIK

Other exemplary anti-CD38 antibodies that may be used in the methods of the invention include those described in Int. Pat. Publ. No. WO05/103083, Intl. Pat. Publ. No. WO06/125640, Intl. Pat. Publ. No. WO07/042309, Intl. Pat. Publ. No. WO08/047242 or Intl. Pat. Publ. No. WO14/178820.

In some embodiments, the antibody that specifically binds CD38 is daratumumab or a biosimilar thereof.

“Biosimilar” (of an approved reference product/biological drug) refers to a biological product that is highly similar to the reference product notwithstanding minor differences in clinically inactive components with no clinically meaningful differences between the biosimilar and the reference product in terms of safety, purity and potency, based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biosimilar. The biosimilar may be an interchangeable product that may be substituted for the reference product at the pharmacy without the intervention of the prescribing healthcare professional. To meet the additional standard of “interchangeability,” the biosimilar is to be expected to produce the same clinical result as the reference product in any given patient and, if the biosimilar is administered more than once to an individual, the risk in terms of safety or diminished efficacy of alternating or switching between the use of the biosimilar and the reference product is not greater than the risk of using the reference product without such alternation or switch. The biosimilar utilizes the same mechanisms of action for the proposed conditions of use to the extend the mechanisms are known for the reference product. The condition or conditions of use prescribed, recommended, or suggested in the labeling proposed for the biosimilar have been previously approved for the reference product. The route of administration, the dosage form, and/or the strength of the biosimilar are the same as those of the reference product and the biosimilar is manufactured, processed, packed or held in a facility that meets standards designed to assure that the biosimilar continues to be safe, pure and potent. The biosimilar may include minor modifications in the amino acid sequence when compared to the reference product, such as N- or C-terminal truncations that are not expected to change the biosimilar performance. The reference product may be approved in at least one of the U.S., Europe, or Japan.

In some embodiments, the antibody that specifically binds CD38 is a non-agonistic antibody.

A non-agonistic antibody that specifically binds CD38 refers to an antibody which upon binding to CD38 does not induce significant proliferation of a sample of peripheral blood mononuclear cells in vitro when compared to the proliferation induced by an isotype control antibody or medium alone.

In some embodiments, the non-agonistic antibody that specifically binds CD38 induces proliferation of peripheral blood mononuclear cells (PBMCs) in a statistically insignificant manner. PBMC proliferation may be assessed by isolating PBMCs from healthy donors and culturing the cells at 1×10⁵ cells/well in flat bottom 96-well plates in the presence or absence of a test antibody in 200 μl RPMI. After a four-day incubation at 37° C., 30 μl ³H-thymidine (16.7 μCi/ml) may be added, and culture may be continued overnight. ³H-thymidine incorporation may be assessed using a Packard Cobra gamma counter (Packard Instruments, Meriden, DT, USA), according to the manufacturer's instructions. Data may be calculated as the mean cpm (±SEM) of PBMCs obtained from several donors. Statistical significance or insignificance between samples cultured in the presence or absence of the test antibody is calculated using standard methods.

Antibodies that specifically bind CD38 that can be used in the methods of the invention may also be selected de novo from, e.g., a phage display library, where the phage is engineered to express human immunoglobulins or portions thereof such as Fabs, single chain antibodies (scFv), or unpaired or paired antibody variable regions (Knappik et al., J Mol Biol 296:57-86, 2000; Krebs et al., J Immunol Meth 254:67-84, 2001; Vaughan et al., Nature Biotechnology 14:309-314, 1996; Sheets et al., PITAS (USA) 95:6157-6162, 1998; Hoogenboom and Winter, J Mol Biol 227:381, 1991; Marks et al., J Mol Biol 222:581, 1991). CD38 binding variable domains may be isolated from e.g., phage display libraries expressing antibody heavy and light chain variable regions as fusion proteins with bacteriophage pIX coat protein as described in Shi et al., J. Mol. Biol. 397:385-96, 2010 and Intl. Pat. Publ. No. WO09/085462). The antibody libraries may be screened for binding to CD38 extracellular domain, the obtained positive clones further characterized, Fabs isolated from the clone lysates, and subsequently cloned as full length antibodies. Such phage display methods for isolating human antibodies are established in the art. See for example: U.S. Pat. Nos. 5,223,409, 5,403,484, 5,571,698, 5,427,908, 5,580,717, 5,969,108, 6,172,197, 5,885,793, 6,521,404, 6,544,731, 6,555,313, 6,582,915, and 6,593,081.

Antibodies may be evaluated for their competition with a reference antibody, for example DARZALEX™ (daratumumab) having the VH of SEQ ID NO: 4 and the VL of SEQ ID NO: 5 for binding to CD38 using well known in vitro methods. In an exemplary method, CHO cells recombinantly expressing CD38 may be incubated with an unlabeled reference antibody for 15 min at 4° C., followed by incubation with an excess of a fluorescently labeled test antibody for 45 min at 4° C. After washing in PBS/BSA, fluorescence may be measured by flow cytometry using standard methods. In another exemplary method, the extracellular domain of CD38 may be coated on the surface of an ELISA plate. Excess of an unlabeled reference antibody may be added for about 15 minutes and subsequently a biotinylated test antibody may be added. After washes in PBS/Tween, binding of the biotinylated test antibody may be detected using horseradish peroxidase (HRP)-conjugated streptavidin and the signal detected using standard methods. It is readily apparent that in the competition assays, the reference antibody may be labelled and the test antibody unlabeled. The test antibody competes with the reference antibody when the reference antibody inhibits binding of the test antibody, or the test antibody inhibits binding of the reference antibody to CD38 by at least 80%, for example 81%, 82%, 83%, 84%, 85%, 86%, 87%, 88%, 89%, 90%, 91%, 92%, 93%, 94%, 95%, 96%, 97%, 98%, 99% or 100%. The epitope of the test antibody may further be defined for example by peptide mapping or hydrogen/deuterium protection assays using known methods, or by crystal structure determination.

Antibodies binding to the region SKRNIQFSCKNIYR (SEQ ID NO: 2) and the region EKVQTLEAWVIHGG (SEQ ID NO: 3) of CD38 (SEQ ID NO: 1) may be generated for example by immunizing mice with peptides having the amino acid sequences shown in SEQ ID NOs: 2 and 3 using standard methods and those described herein, and characterizing the obtained antibodies for binding to the peptides using for example known ELISA or mutagenesis studies.

Antibodies that are substantially identical to the antibody comprising the VH of SEQ ID NO: 4 and the VL of SEQ ID NO: 5 may be used in the methods of the invention. The term “substantially identical” as used herein means that the antibody VH or VL amino acid sequences being compared are identical or have “insubstantial differences”. Insubstantial differences are substitutions of 1, 2, 3, 4, 5, 6, 7, 8, 9, 10, 11, 12, 13, 14, or 15 amino acids in the antibody VL and/or VL that do not adversely affect antibody properties. Percent identity may be determined for example by pairwise alignment using the default settings of the AlignX module of Vector NTI v.9.0.0 (Invitrogen, Carlsbad, CA). The protein sequences of the present invention may be used as a query sequence to perform a search against public or patent databases to, for example, identify related sequences. Exemplary programs used to perform such searches are the XBLAST or BLASTP programs (http_//www_ncbi_nlm/nih_gov), or the GenomeQuest™ (GenomeQuest, Westborough, MA) suite using the default settings. Exemplary substitutions that may be made to the antibodies that specifically bind CD38 used in the methods of the invention are for example conservative substitutions with an amino acid having similar charge, hydrophobic, or stereochemical characteristics. Conservative substitutions may also be made to improve antibody properties, for example stability or affinity, or to improve antibody effector functions. 1, 2, 3, 4, 5, 6, 7, 8, 9, 10, 11, 12, 13, 14, or 15 amino acid substitutions may be made for example to the heavy or the light chain of the antibodies that specifically binds CD38. Furthermore, any native residue in the heavy or light chain may also be substituted with alanine, as has been previously described for alanine scanning mutagenesis (MacLennan et al., Acta Physiol Scand Suppl 643:55-67, 1998; Sasaki et al., Adv Biophys 35:1-24, 1998). Desired amino acid substitutions may be determined by those skilled in the art at the time such substitutions are desired. Amino acid substitutions may be done for example by PCR mutagenesis (U.S. Pat. No. 4,683,195). Libraries of variants may be generated using well known methods, for example using random (NNK) or non-random codons, for example DVK codons, which encode 11 amino acids (Ala, Cys, Asp, Glu, Gly, Lys, Asn, Arg, Ser, Tyr, Trp) and screening the libraries for variants with desired properties. The generated variants may be tested for their binding to CD38, their ability to induce ADCC, ADCP or apoptosis, or modulate CD38 enzymatic activity in vitro using methods described herein.

“Conservative modifications” refer to amino acid modifications that do not significantly affect or alter the binding characteristics of the antibody containing the amino acid sequences. Conservative modifications include amino acid substitutions, additions and deletions. Conservative substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al., (1988) Acta Physiol Scand Suppl 643:55-67; Sasaki et al., (1988) Adv Biophys 35:1-24). Amino acid substitutions to the antibodies of the invention may be made by known methods for example by PCR mutagenesis (U.S. Pat. No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting antibody variants may be tested for their characteristics using assays described herein.

In some embodiments, the antibody may bind CD38 with a dissociation constant (K_{D}) of less than about 1×10⁻⁷ M, 1×10⁻⁸ M, 1×10⁻⁹ M, 1×10⁻¹⁰ M, 1×10⁻¹¹ M, 1×10⁻¹² M, 1×10⁻¹³ M, 1×10⁻¹⁴ M or 1×10⁻¹⁵, as determined by surface plasmon resonance or the Kinexa method, as practiced by those of skill in the art. In some embodiments, the antibody binds human CD38 with a K_{D} of less than about 1×10⁻⁸ M. In some embodiments, the antibody binds human CD38 with a K_{D} of less than about 1×10⁻⁹ M.

KinExA instrumentation, ELISA or competitive binding assays known to those skilled in the art. The measured affinity of a particular antibody/CD38 interaction may vary if measured under different conditions (e.g., osmolarity, pH). Thus, measurements of affinity and other binding parameters (e.g., K_{D}), Kₒₙ, K_{off}) are typically made with standardized conditions and a standardized buffer, such as the buffer described herein. Skilled in the art will appreciate that the internal error for affinity measurements for example using Biacore 3000 or ProteOn (measured as standard deviation, SD) may typically be within 5-33% for measurements within the typical limits of detection. Therefore the term “about” in the context of K_{D} reflects the typical standard deviation in the assay. For example, the typical SD for a K_{D} of 1×10⁻⁹ M is up to ±0.33×10⁻⁹ M.

In some embodiments, the antibody that specifically binds CD38 is an IgG1, IgG2, IgG3 or IgG4 isotype.

In some embodiments, the antibody that specifically binds CD38 is an IgG1 isotype.

In some embodiments, the antibody that specifically binds CD38 is an IgG2 isotype.

In some embodiments, the antibody that specifically binds CD38 is an IgG3isotype.

In some embodiments, the antibody that specifically binds CD38 is an IgG4 isotype.

The Fc portion of the antibody may mediate antibody effector functions such as antibody-dependent cell-mediated cytotoxicity (ADCC), antibody-dependent cellular phagocytosis (ADCP) or complement dependent cytotoxicity (CDC). Such function may be mediated by binding of an Fc effector domain(s) to an Fc receptor on an immune cell with phagocytic or lytic activity or by binding of an Fc effector domain(s) to components of the complement system. Typically, the effect(s) mediated by the Fc-binding cells or complement components result in inhibition and/or depletion of target cells, for example CD38-expressing cells. Human IgG isotypes IgG1, IgG2, IgG3 and IgG4 exhibit differential capacity for effector functions. ADCC may be mediated by IgG1 and IgG3, ADCP may be mediated by IgG1, IgG2, IgG3 and IgG4, and CDC may be mediated by IgG1 and IgG3.

“Antibody-dependent cellular cytotoxicity”, “antibody-dependent cell-mediated cytotoxicity” or “ADCC” is a mechanism for inducing cell death that depends upon the interaction of antibody-coated target cells with effector cells possessing lytic activity, such as natural killer cells, monocytes, macrophages and neutrophils via Fc gamma receptors (FcγR) expressed on effector cells. For example, NK cells express FcγRIIIa, whereas monocytes express FcγRI, FcγRII and FcγRIIIa. Death of the antibody-coated target cell, such as CD38-expressing cells, occurs as a result of effector cell activity through the secretion of membrane pore-forming proteins and proteases. To assess ADCC activity of an antibody that specifically binds CD38, the antibody may be added to CD38-expressing cells in combination with immune effector cells, which may be activated by the antigen antibody complexes resulting in cytolysis of the target cell. Cytolysis is generally detected by the release of label (e.g. radioactive substrates, fluorescent dyes or natural intracellular proteins) from the lysed cells. Exemplary effector cells for such assays include peripheral blood mononuclear cells (PBMC) and NK cells. Exemplary target cells include B cells expressing CD38. In an exemplary assay, target cells are labeled with 20 μCi of ⁵¹Cr for 2 hours and washed extensively. Cell concentration of the target cells may be adjusted to 1×10⁶ cells/ml, and antibodies specifically binding CD38 at various concentrations are added. Assays are started by adding target cells at an effector:target cell ratio of 40:1. After incubation for 3 hr at 37° C. assays are stopped by centrifugation, and ⁵¹Cr release from lysed cells are measured in a scintillation counter. Percentage of cellular cytotoxicity may be calculated as % maximal lysis which may be induced by adding 3% perchloric acid to target cells.

“Antibody-dependent cellular phagocytosis” (“ADCP”) refers to a mechanism of elimination of antibody-coated target cells by internalization by phagocytic cells, such as macrophages or dendritic cells. ADCP may be evaluated using CD38-positive cells engineered to express GFP or other labeled molecule as target cells. Effctor:target cell ratio may be for example 4:1. Effector cells may be incubated with target cells for 4 hours with or without antibodies that specifically bind CD38. After incubation, cells may be detached using accutase. Macrophages may be identified with anti-CD11b and anti-CD14 antibodies coupled to a fluorescent label, and percent phagocytosis may be determined based on % GFP fluorescent in the CD11 CD14 macrophages using standard methods.

“Complement-dependent cytotoxicity”, or “CDC”, refers to a mechanism for inducing cell death in which an Fc effector domain of a target-bound antibody binds and activates complement component C1q which in turn activates the complement cascade leading to target cell death. Activation of complement may also result in deposition of complement components on the target cell surface that facilitate ADCC by binding complement receptors (e.g., CR3) on leukocytes.

The ability of monoclonal antibodies to induce ADCC may be enhanced by engineering their oligosaccharide component. Human IgG1 or IgG3 are N-glycosylated at Asn297 with most the glycans in the well-known biantennary G0, G0F, G1, G1F, G2 or G2F forms. Antibodies produced by non-engineered CHO cells typically have a glycan fucose content of about at least 85%. The removal of the core fucose from the biantennary complex-type oligosaccharides attached to the Fc regions enhances the ADCC of antibodies via improved FcγRIIIa binding without altering antigen binding or CDC activity. Such mAbs may be achieved using different methods reported to lead to the successful expression of relatively high defucosylated antibodies bearing the biantennary complex-type of Fc oligosaccharides such as control of culture osmolality (Konno et al., Cytotechnology 64:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2 (4), 2010; Epub ahead of print; PMID: 20562582), application of a rat hybridoma cell line YB2/0 as the host cell line (Shinkawa et al., J Biol Chem 278:3466-3473, 2003), introduction of small interfering RNA specifically against the α 1,6-fucosyltrasferase (FUT8) gene (Mori et al., Biotechnol Bioeng88: 901-908, 2004), or coexpression of β-1,4-N-acetylglucosaminyltransferase III and Golgi α-mannosidase II or a potent alpha-mannosidase I inhibitor, kifunensine (Ferrara et al., J Biol Chem 281: 5032-5036, 2006, Ferrara et al., Biotechnol Bioeng 93:851-861, 2006; Xhou et al., Biotechnol Bioeng 99:652-65, 2008). ADCC elicited by anti-CD38 antibodies used in the methods of the invention may also be enhanced by certain substitutions in the antibody Fc. Exemplary substitutions are for example substitutions at amino acid positions 256, 290, 298, 312, 356, 330, 333, 334, 360, 378 or 430 (residue numbering according to the EU index) as described in U.S. Pat. No. 6,737,056.

In some embodiments, the antibody that specifically binds CD38 comprises a substitution in the antibody Fc.

In some embodiments, the antibody that specifically binds CD38 comprises a substitution in the antibody Fc at amino acid positions 256, 290, 298, 312, 356, 330, 333, 334, 360, 378 or 430 (residue numbering according to the EU index).

In some embodiments, the antibody that specifically binds CD38 has a biantennary glycan structure with fucose content of about between 0% to about 15%, for example 15%, 14%, 13%, 12%, 11% 10%, 9%, 8%, 7%, 6%, 5%, 4%, 3%, 2%, 1% or 0%.

In some embodiments, the antibody that specifically binds CD38 has a biantennary glycan structure with fucose content of about 50%, 40%, 45%, 40%, 35%, 30%, 25%, 20%, 15%, 14%, 13%, 12%, 11% 10%, 9%, 8%, 7%, 6%, 5%, 4%, 3%, 2%, 1% or 0%

Substitutions in the Fc and reduced fucose content may enhance the ADCC activity of the antibody that specifically binds CD38.

“Fucose content” means the amount of the fucose monosaccharide within the sugar chain at Asn297. The relative amount of fucose is the percentage of fucose-containing structures related to all glycostructures. These may be characterized and quantified by multiple methods, for example: 1) using MALDI-TOF of N-glycosidase F treated sample (e.g. complex, hybrid and oligo- and high-mannose structures) as described in Intl. Pat. Publ. No. WO2008/077546; 2) by enzymatic release of the Asn297 glycans with subsequent derivatization and detection/quantitation by HPLC (UPLC) with fluorescence detection and/or HPLC-MS (UPLC-MS); 3) intact protein analysis of the native or reduced mAb, with or without treatment of the Asn297 glycans with Endo S or other enzyme that cleaves between the first and the second GlcNAc monosaccharides, leaving the fucose attached to the first GlcNAc; 4) digestion of the mAb to constituent peptides by enzymatic digestion (e.g., trypsin or endopeptidase Lys-C), and subsequent separation, detection and quantitation by HPLC-MS (UPLC-MS) or 5) separation of the mAb oligosaccharides from the mAb protein by specific enzymatic deglycosylation with PNGase F at Asn 297. The oligosaccharides released may be labeled with a fluorophore, separated and identified by various complementary techniques which allow: fine characterization of the glycan structures by matrix-assisted laser desorption ionization (MALDI) mass spectrometry by comparison of the experimental masses with the theoretical masses, determination of the degree of sialylation by ion exchange HPLC (GlycoSep C), separation and quantification of the oligosacharride forms according to hydrophilicity criteria by normal-phase HPLC (GlycoSep N), and separation and quantification of the oligosaccharides by high performance capillary electrophoresis-laser induced fluorescence (HPCE-LIF).

“Low fucose” or “low fucose content” as used herein refers to antibodies with fucose content of about 0%-15%.

“Normal fucose” or ‘normal fucose content” as used herein refers to antibodies with fucose content of about over 50%, typically about over 60%, 70%, 80% or over 85%.

The antibody that specifically binds CD38 used in the methods of the invention may induce killing of CD38-expressing IgE producing cells by apoptosis. Methods for evaluating apoptosis are well known, and include for example annexin IV staining using standard methods. The antibodies that specifically bind human CD38 may induce apoptosis in about 20%, 25%, 30%, 35%, 40%, 45%, 50%, 55%, 60%, 65%, 70%, 75%, 80%, 85%, 90%, 95% or 100% of cells.

In some embodiments, the antibody that specifically binds human CD38 is a bispecific antibody. The VL and/or the VH regions of the existing antibodies that specifically bind CD38 or the VL and VH regions identified de novo as described herein may be engineered into bispecific full length antibodies. Such bispecific antibodies may be made by modulating the CH3 interactions between the monospecific antibody heavy chains to form bispecific antibodies using technologies such as those described in U.S. Pat. No. 7,695,936; Intl. Pat. Publ. No. WO04/111233; U.S. Pat. Publ. No. US2010/0015133; U.S. Pat. Publ. No. US2007/0287170; Intl. Pat. Publ. No. WO2008/119353; U.S. Pat. Publ. No. US2009/0182127; U.S. Pat. Publ. No. US2010/0286374; U.S. Pat. Publ. No. US2011/0123532; Intl. Pat. Publ. No. WO2011/131746; Int. Pat. Publ. No. WO2011/143545; or U.S. Pat. Publ. No. US2012/0149876. Additional bispecific structures into which the VL and/or the VH regions of the antibodies of the invention may be incorporated are for example Dual Variable Domain Immunoglobulins (Intl. Pat. Publ. No. WO2009/134776), or structures that include various dimerization domains to connect the two antibody arms with different specificity, such as leucine zipper or collagen dimerization domains (Int. Pat. Publ. No. WO2012/022811, U.S. Pat. Nos. 5,932,448; 6,833,441).

For example, bispecific antibodies may be generated in vitro in a cell-free environment by introducing asymmetrical mutations in the CH3 regions of two monospecific homodimeric antibodies and forming the bispecific heterodimeric antibody from two parental monospecific homodimeric antibodies in reducing conditions to allow disulfide bond isomerization according to methods described in Intl. Pat. Publ. No. WO2011/131746. In the methods, the first monospecific bivalent antibody (e.g., anti-CD38 antibody) and the second monospecific bivalent antibody are engineered to have certain substitutions at the CH3 domain that promote heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl) phosphine (TCEP), L-cysteine and beta-mercaptoethanol, preferably a reducing agent selected from the group consisting of: 2-mercaptoethylamine, dithiothreitol and tris(2-carboxyethyl) phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

Exemplary CH3 mutations that may be used in a first heavy chain and in a second heavy chain of the bispecific antibody are K409R and/or F405L.

The methods of the invention may be used to treat an animal patient belonging to any classification. Examples of such animals include mammals such as humans, rodents, dogs, cats and farm animals.

### Administration/Pharmaceutical Compositions

In the methods of the invention, the antibodies that specifically bind CD38 may be provided in suitable pharmaceutical compositions comprising the antibody and a pharmaceutically acceptable carrier. The carrier may be diluent, adjuvant, excipient, or vehicle with which the antibodies that specifically bind CD38 are administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the molecules or antibodies of the invention in such pharmaceutical formulation may vary widely, i.e., from less than about 0.5%, usually to at least about 1% to as much as 15 or 20%, 25%, 30%, 35%, 40%, 45% or 50% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21^{st} Edition, Troy, D. B. ed., Lipincott Williams and Wilkins, Philadelphia, PA 2006, Part 5, Pharmaceutical Manufacturing pp 691-1092, see especially pp. 958-989.

The mode of administration of the antibodies that specifically bind CD38 may be any suitable route such as parenteral administration, e.g., intradermal, intramuscular, intraperitoneal, intravenous or subcutaneous, pulmonary, transmucosal (oral, intranasal, intravaginal, rectal) or other means appreciated by the skilled artisan, as well known in the art.

The antibodies that specifically bind CD38 may be administered to a subject by any suitable route, for example parentally by intravenous (i.v.) infusion or bolus injection, intramuscularly or subcutaneously or intraperitoneally. i.v. infusion may be given over for example 15, 30, 60, 90, 120, 180, or 240 minutes, or from 1, 2, 3, 4, 5, 6, 7, 8, 9, 10, 11 or 12 hours.

The dose given to a subject is sufficient to alleviate or at least partially arrest the disease being treated (“therapeutically effective amount”) and may be sometimes 0.005 mg to about 100 mg/kg, e.g. about 0.05 mg to about 30 mg/kg or about 5 mg to about 25 mg/kg, or about 4 mg/kg, about 8 mg/kg, about 16 mg/kg or about 24 mg/kg, or for example about 1, 2, 3, 4, 5, 6, 7, 8, 9 or 10 mg/kg, but may even higher, for example about 15, 16, 17, 18, 19, 20, 21, 22, 23, 24, 25, 30, 40, 50, 60, 70, 80, 90 or 100 mg/kg.

A fixed unit dose may also be given, for example, 50, 100, 200, 500 or 1000 mg, or the dose may be based on the patient's surface area, e.g., 500, 400, 300, 250, 200, or 100 mg/m². Usually between 1 and 8 doses, (e.g., 1, 2, 3, 4, 5, 6, 7 or 8) may be administered, but 9, 10, 11, 12, 13, 14, 15, 16, 17, 18, 19, 20 or more doses may be given.

The administration of the antibodies that specifically bind CD38 in the methods of the invention may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose. For example, the antibodies that specifically bind CD38 may be administered at 8 mg/kg or at 16 mg/kg at weekly interval for 8 weeks, followed by administration at 8 mg/kg or at 16 mg/kg every two weeks for an additional 16 weeks, followed by administration at 8 mg/kg or at 16 mg/kg every four weeks by intravenous infusion.

The antibodies that specifically bind CD38 may be administered as maintenance therapy, such as, e.g., once a week for a period of 6 months or more.

For example, the antibodies that specifically bind CD38 may be provided as a daily dosage in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

The antibodies that specifically bind CD38 may be lyophilized for storage and reconstituted in a suitable carrier prior to use. This technique has been shown to be effective with conventional protein preparations and well known lyophilization and reconstitution techniques can be employed.

The antibodies that specifically bind CD38 may be administered prophylactically to reduce the risk of developing an IgE-mediated disease.

The antibodies that specifically bind CD38 may also be administered within seconds, minutes, or hours following a subject's exposure to an allergen, or when at least one symptom of the IgE-mediated disease is present in a subject. Thus, the methods used herein are useful in the treatment of both an acute exposure to an allergen and chronic (e.g., seasonal) exposure to an allergen.

The antibodies that specifically bind CD38 may be administered in combination with a second therapeutic agent.

The second therapeutic agent may be a standard of care treatment for the IgE-mediated disease, such as allergy, allergic asthma, urticarial, angioedema, autoimmune and inflammatory diseases.

Subcutaneous administration of pharmaceutical compositions comprising an antibody that specifically binds CD38 and a hyaluronidase

The antibody that specifically binds CD38 may be administered as a pharmaceutical composition comprising the antibody that specifically binds CD38 and a hyaluronidase subcutaneously.

The concentration of the antibody that specifically binds CD38 in the pharmaceutical composition administered subcutaneously may be about 20 mg/ml.

The pharmaceutical composition administered subcutaneously may comprise between about 1,200 mg-1,800 mg of the antibody that specifically binds CD38.

The pharmaceutical composition administered subcutaneously may comprise about 1,200 mg of the antibody that specifically binds CD38.

The pharmaceutical composition administered subcutaneously may comprise about 1,600 mg of the antibody that specifically binds CD38.

The pharmaceutical composition administered subcutaneously may comprise about 1,800 mg of the antibody that specifically binds CD38.

The pharmaceutical composition administered subcutaneously may comprise between about 30,000 U-45,000 U of the hyaluronidase.

The pharmaceutical composition administered subcutaneously may comprise about 1,200 mg of the antibody that specifically binds CD38 and about 30,000 U of the hyaluronidase.

The pharmaceutical composition administered subcutaneously may comprise about 1,800 mg of the antibody that specifically binds CD38 and about 45,000 U of the hyaluronidase.

The pharmaceutical composition administered subcutaneously may comprise about 1,600 mg of the antibody that specifically binds CD38 and about 30,000 U of the hyaluronidase.

The pharmaceutical composition administered subcutaneously may comprise about 1,600 mg of the antibody that specifically binds CD38 and about 45,000 U of the hyaluronidase.

The pharmaceutical composition administered subcutaneously may comprise the hyaluronidase rHuPH20 having the amino acid sequence of SEQ ID NO: 22.

rHuPH20 is a recombinant hyaluronidase (HYLENEX® recombinant) and is described in Int. Pat. Publ. No. WO2004/078140.

Hyaluronidase is an enzyme that degrades hyaluronic acid (EC 3.2.1.35) and lowers the viscosity of hyaluronan in the extracellular matrix, thereby increasing tissue permeability.

SEQ ID NO: 22
MGVLKFKHIFFRSFVKSSGVSQIVFTFLLIPCCLTLNFRA
PPVIPNVPFLWAWNAPSEFCLGKFDEPLDMSLFSFIGSPR
INATGQGVTIFYVDRLGYYPYIDSITGVTVNGGIPQKISL
QDHLDKAKKDITFYMPVDNLGMAVIDWEEWRPTWARNWKP
KDVYKNRSIELVQQQNVQLSLTEATEKAKQEFEKAGKDFL
VETIKLGKLLRPNHLWGYYLFPDCYNHHYKKPGYNGSCFN
VEIKRNDDLSWLWNESTALYPSIYLNTQQSPVAATLYVRN
RVREAIRVSKIPDAKSPLPVFAYTRIVFTDQVLKFLSQDE
LVYTFGETVALGASGIVIWGTLSIMRSMKSCLLLDNYMET
ILNPYIINVTLAAKMCSQVLCQEQGVCIRKNWNSSDYLHL
NPDNFAIQLEKGGKFTVRGKPTLEDLEQFSEKFYCSCYST
LSCKEKADVKDTDAVDVCIADGVCIDAFLKPPMETEEPQI
FYNASPSTLSATMFIVSILFLIISSVASL

The administration of the pharmaceutical composition comprising the antibody that specifically binds CD38 and the hyaluronidase may be repeated after one day, two days, three days, four days, five days, six days, one week, two weeks, three weeks, four weeks, five weeks, six weeks, seven weeks, two months, three months, four months, five months, six months or longer. Repeated courses of treatment are also possible, as is chronic administration. The repeated administration may be at the same dose or at a different dose. For example, the pharmaceutical composition comprising the antibody that specifically binds CD38 and the hyaluronidase may be administered once weekly for eight weeks, followed by once in two weeks for 16 weeks, followed by once in four weeks. The pharmaceutical compositions to be administered may comprise about 1,200 mg of the antibody that specifically binds CD38 and about 30,000 U of hyaluronidase, wherein the concentration of the antibody that specifically binds CD38 in the pharmaceutical composition is about 20 mg/ml. The pharmaceutical compositions to be administered may comprise about 1,800 mg of the antibody that specifically binds CD38 and about 45,000 U of hyaluronidase. The pharmaceutical compositions to be administered may comprise about 1,600 mg of the antibody that specifically binds CD38 and about 30,000 U of hyaluronidase. The pharmaceutical compositions to be administered may comprise about 1,600 mg of the antibody that specifically binds CD38 and about 45,000 U of hyaluronidase.

The pharmaceutical composition comprising the antibody that specifically binds CD38 and the hyaluronidase may be administered subcutaneously to the abdominal region.

The pharmaceutical composition comprising the antibody that specifically binds CD38 and the hyaluronidase may be administered in a total volume of about 80 ml, 90 ml, 100 ml, 110 ml or 120 ml.

For administration, 20 mg/ml of the antibody that specifically binds CD38 antibody in 25 mM sodium acetate, 60 mM sodium chloride, 140 mM D-mannitol, 0.04% polysorbate 20, pH 5.5 may be mixed with rHuPH20, 1.0 mg/mL (75-150 kU/mL) in 10 mM L-Histidine, 130 mM NaCl, 10 mM L-Methionine, 0.02% Polysorbate 80, pH 6.5 prior to administration of the mixture to a subject.

While having described the invention in general terms, the embodiments of the invention will be further disclosed in the following examples that should not be construed as limiting the scope of the claims.

### Example 1. Daratumumab Reduces IgE Levels in Allergic Patients

The effect of DARZALEX™ (dratumumab) treatment on total and specific IgE levels via the depletion of IgE-producing plasma cells was evaluated. This pilot study could demonstrate the potential value of daratumumab in management of patients with severe IgE-mediated disease.

Residual blood samples were collected from patients with multiple myeloma (MM) treated with daratumumab monotherapy or daratumumab plus lenalidomide-dexamethsone. Routine blood analysis was performed at baseline and after every four weeks of daratumumab treatment.

In the baseline samples, total IgE was determined and an ImmunoCAP Phadiatop (ThermoFisher, Uppsala, Sweden) was performed, according to manufacturer's instructions, to indicate sensitization to common inhalation allergens. If the Phadiatop was positive, specific IgE (sIgE) against birch pollen, timothy grass pollen and house dust mite was determined using ImmunoCAP (ThermoFisher, Uppsala, Sweden). These are among the most frequently recognized inhalant allergens in the Netherlands.

Determination of sIgE was performed by the ImmunoCAP technique for both the Phadiatop as the specific/total IgE according to the manufacturer's instructions (Thermo Fisher Scientific, Uppsala Sweden). This technique uses allergens covalently coupled to a solid phase matrix consisting of a cellulose derivative enclosed in a capsule. Analysis of binding of sIgE to the coated allergens is quantified by enzyme-labelled anti-IgE using fluorescence as read-out. sIgE levels≥0.35 kU/L were defined as positive test results.

### Catalogue Numbers:

Total IgE: 14-4509-01
Phadiatop: 14-4405-35
Birch slgE: 14-4102-01 (t3)
Timothy grass pollen slgE: 14-4100-01 (g6)
House dust mite slgE: 14-4107-01 (d1)

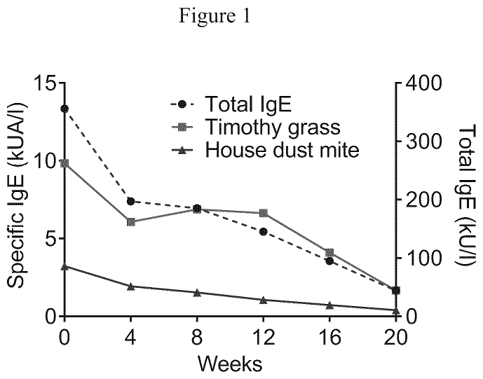

A total of eight patients were included; five treated with daratumumab monotherapy and three with daratumumab plus lenalidomide-dexamethasone. Four patients had a detectable IgE (≥2 kU/L) at baseline and are listed in Table 1. Only for the first patient (patient 1), specific IgE levels were elevated above reference levels and specific IgE against inhalant allergens were detected. Additional samples from patient 1 at week 4, 8, 12, 16 and 20 were analyzed, demonstrating a decrease of more than 80% in both total IgE and specific IgE levels for timothy grass pollen and house dust mite after 20 weeks (Table 2 and FIG. 1). The other three patients with detectable IgE levels also demonstrated a decrease in total IgE after eight weeks of treatment. For patient 2, total IgE levels decreased with 88% (41 to 5 kU/L). For the other two patients, baseline IgE levels were very low and dropped below detection limit after eight weeks. Percentage of benign and malignant plasma cells were reduced with daratumumab treatment in all patients (Table 3). Patient 1 M-protein and free kappa chain levels were reduced over time (Table 4).

In conclusion, this proof of concept demonstrates that levels of total and specific IgE gradually decrease during daratumumab treatment in a single patient sensitized to two common inhalant allergens. This patient was co-treated with lenalidomide and dexamethasone. The effect of lenalidomide on IgE levels is unknown, but glucocorticoids are known to increase IgE synthesis (Hemady Z et al., J Allergy Clin Immunol 1985; 75:304-12. doi: 10.1016/0091-6749 (85) 90062-4; Wu C Y et al., J Clin Invest 1991; 87:870-7. doi: 10.1172/JCI115092). IgE depletion by omalizumab treatment resulted in clinical improvement of disease, as well as increased quality of life and reduced socio-economic burden of disease, both in clinical trials as well as daily practice studies (Abraham I et al., Allergy 2015: n/a-n/a. doi: 10.1111/all.12815). The effect of plasma cell depletion by daratumumab on clinical parameters of allergy has yet to be investigated.

TABLE 1

| | | Total | | | Tim.grass | HDM |
|---|---|---|---|---|---|---|
| | | IgE | | Birch sIgE | sIgE | sIgE |
| Patient | Study | (kU/L) | Phadiatop | (kU/L) | (kU/L) | (kU/L) |
| 1 | D + LD* | 356 | Positive | Negative | 9.83 | 3.23 |
| 2 | D + LD* | 41 | Negative | — | — | — |
| 3 | D** | 3 | Negative | — | — | — |
| 4 | D + LD* | 7 | Negative | — | — | — |

Abbreviations:
D: daratumumab monotherapy,
D + L: daratumumab plus lenalidomide-dexamethasone,
Birch: birch pollen,
Tim.grass: timothy grass pollen,
HDM: house dust mite
Reference values:
Total IgE: <100 kU/L;
ImmunoCAP sIgE birch/tim.grass/hdm: <0.35 kU/L
*Patient was enrolled in the GEN503 study and received daratumumab 16 mg/kg and lenalidomide 25 mg.
**Patient was in the GEN501 phase II study and received daratumumab 16 mg/kg.

TABLE 2

| Week | W0 | W4 | W8 | W12 | W16 | W20 |
|---|---|---|---|---|---|---|
| Total IgE | 356 | 197 | 185 | 145 | 95 | 44 |
| Tim.grass | 9.83 | 6.07 | 6.88 | 6.63 | 4.1 | 1.67 |
| HDM | 3.23 | 1.93 | 1.54 | 1.06 | 0.73 | 0.4 |

Abbreviations:
Tim.grass: timothy grass pollen,
HDM: house dust mite
Reference values:
Total IgE: <100 kU/L;
ImmunoCAP sIgE tim.grass/hdm: <0.35 kU/L

TABLE 3

| | | Re- | | | | |
|---|---|---|---|---|---|---|
| | | sponse | % benign | | % malignant | |
| | | to | plasma cells | | plasma cells | |
| | Start | treat- | Pre- | After | Pre- | After |
| Patient | treatment | ment | treatment | W8/12 | treatment | W8/12 |
| 1 | April 2014 | CR | 0.420 | 0.240 | 0.226 | 0.015 |
| 2 | April 2014 | CR | 0.168 | 0.033 | 2.485 | 0.539 |
| 3 | April 2014 | PD | 0.032 | 0 | 2.543 | 0.283 |
| 4 | June 2014 | CR | 0.055 | 0 | 6.214 | 0.025 |

CR: complete response,
PD: progressive disease.

TABLE 4

| | M-protein levels | Free kappa |
|---|---|---|
| Time | quantitative | chains |
| point | (g/l) | (mg/L) |
| W0 | 16 | 52.96 |
| W4 | 5 | 14.52 |
| W8 | 3 | 16.40 |
| W12 | Not quantifiable | 14.28 |
| W16 | Not quantifiable | 13.58 |
| W20 | Not quantifiable | 12.69 |

What is claimed is:

1. A method of reducing an immunoglobulin type E (IgE) level in a subject in need thereof, comprising administering to the subject an antibody that specifically binds CD38 at about 5 mg/kg to about 25 mg/kg, wherein the antibody that specifically binds CD38 comprises a heavy chain variable region (VH) of SEQ ID NO: 4 and a light chain variable region (VL) of SEQ ID NO: 5.

2. The method of claim 1, wherein the antibody that specifically binds CD38 is an IgG1, IgG2, IgG3 or IgG4 isotype.

3. The method of claim 1, wherein the antibody that specifically binds CD38 is an IgG1 isotype.

4. The method of claim 1, wherein the antibody that specifically binds CD38 comprises a heavy chain of SEQ ID NO: 12 and a light chain of SEQ ID NO: 13.

5. The method of claim 1, wherein the antibody that specifically binds CD38 is daratumumab.

6. The method of claim 1, wherein the antibody that specifically binds CD38 is administered intravenously.

7. The method of claim 1, wherein the method reduces the IgE level in the subject by about 40% to about 90%.

8. The method of claim 1, wherein the method treats an IgE-mediated disease or reduces a risk of developing an IgE-mediated disease.

9. The method of claim 8, wherein the IgE-mediated disease is acute.

10. The method of claim 8, wherein the IgE-mediated disease is chronic.

11. The method of claim 8, wherein the IgE-mediated disease is allergic asthma, urticaria, angioedema, food allergy, an allergic response, atopic dermatitis, anaphylaxis, cutaneous mastocystosis, allergic rhinitis, nasal polyposis, Kimura's disease, eosinophilic otitis media, eosinophilic gastroenteritis, latex allergy, bronchopulmonary allergic aspergillosis, bullous pemphigoid, systemic lupus erythematosus, lupus, rheumatoid arthritis or an autoimmune disease.

12. The method of claim 8, wherein the IgE-mediated disease is an autoimmune disease.

13. The method of claim 8, wherein the IgE-mediated disease is an allergic response.

14. The method of claim 8, wherein the antibody that specifically binds CD38 is administered to the subject following an exposure to an allergen.

15. The method of claim 8, wherein the antibody that specifically binds CD38 is administered to the subject prophylactically to reduce the risk of developing the IgE-mediated disease.

16. The method of claim 1, wherein the antibody that specifically binds CD38 is administered with a second therapeutic agent.

17. The method of claim 16, wherein the second therapeutic agent is a standard of care treatment for an IgE-mediated disease.

18. The method of claim 1, wherein the antibody that specifically binds CD38 induces killing of IgE producing CD38⁺ cells.

19. The method of claim 18, wherein the antibody that specifically binds CD38 induces killing of IgE producing CD38⁺ cells by antibody-dependent cell-mediated cytotoxicity (ADCC), antibody-dependent cellular phagocytosis (ADCP), complement dependent cytotoxicity (CDC), apoptosis, or modulation of CD38 enzymatic activity.

20. The method of claim 19, wherein the antibody that specifically binds CD38 induces killing of IgE producing CD38⁺ cells by ADCC.


SEQUENCE LISTING (22 sequences)

SEQ ID NO: 1 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 14 aa, Homo sapiens
SKRNIQFSCKNIYR

SEQ ID NO: 3 - protein, 14 aa, Homo sapiens
EKVQTLEAWVIHGG

SEQ ID NO: 4 - protein, 122 aa, synthetic construct [sequence omitted]

SEQ ID NO: 5 - protein, 107 aa, synthetic construct [sequence omitted]

SEQ ID NO: 6 - protein, 5 aa, synthetic construct
  REGION 1..5: note = HCDR1 of daratumumab
SFAMS

SEQ ID NO: 7 - protein, 17 aa, synthetic construct
  REGION 1..17: note = HCDR2 of daratumumab
AISGSGGGTYYADSVKG

SEQ ID NO: 8 - protein, 13 aa, synthetic construct
  REGION 1..13: note = HCDR3 of daratumumab
DKILWFGEPVFDY

SEQ ID NO: 9 - protein, 11 aa, synthetic construct
  REGION 1..11: note = LCDR1 of daratumumab
RASQSVSSYLA

SEQ ID NO: 10 - protein, 7 aa, synthetic construct
  REGION 1..7: note = LCDR2 of daratumumab
DASNRAT

SEQ ID NO: 11 - protein, 10 aa, synthetic construct
  REGION 1..10: note = LCDR3 of daratumumab
QQRSNWPPTF

SEQ ID NO: 12 - protein, 452 aa, synthetic construct
  REGION 1..452: note = HC of daratumumab
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, synthetic construct
  REGION 1..214: note = LC of daratumumab
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 122 aa, synthetic construct
  REGION 1..122: note = mAb 003 VH
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSSAS

SEQ ID NO: 15 - protein, 107 aa, synthetic construct
  REGION 1..107: note = mAb 003 VL
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIK

SEQ ID NO: 16 - protein, 122 aa, synthetic construct
  REGION 1..122: note = mAb 024 VH
EVQLVQSGAEVKKPGESLKISCKGSGYSFSNYWIGWVRQMPGKGLEWMGIIYPHDSDARYSPSFQGQVTFSADKSISTAYLQWSSLKASDTAMYYCARHVGWGSRYWYFDLWGRGTLVTVSS

SEQ ID NO: 17 - protein, 107 aa, synthetic construct
  REGION 1..107: note = mAb 024 VL
EIVLTQSPATLSLSPGERATLSCRASQSVSSYLAWYQQKPGQAPGLLIYDASNRASGIPARFSGSGSGTDFTLTISSLEPEDFAVYYCQQRSNWPLTFGGGTKVEIK

SEQ ID NO: 18 - protein, 120 aa, synthetic construct
  REGION 1..120: note = MOR202 VH
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 19 - protein, 109 aa, synthetic construct
  REGION 1..109: note = MOR202 VL
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 20 - protein, 120 aa, synthetic construct [sequence omitted]

SEQ ID NO: 21 - protein, 107 aa, synthetic construct [sequence omitted]

SEQ ID NO: 22 - protein, 509 aa, synthetic construct
  REGION 1..509: note = recombinant hyaluronidase
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL